(19) United States
(12) Patent Application Publication
(10) Pub. No.: US 2008/0317879 A1
(43) Pub. Date: Dec. 25, 2008

(54) Herb Composition for Asthma Maintenance Therapy and Manufacturing Method Thereof

(76) Inventors: Sung-Ki Jung, Seoul (KR); Hee-Jae Jung, Seoul (KR); Woo-Suck Hwang, Seoul (KR); Jun-Yong Choi, Seoul (KR); Hyun-Su Bae, Seoul (KR); Kyu-Bum Kwack, Seoul (KR); Hong-Yeoul Kim, Seoul (KR)

Correspondence Address: IPLA P.A., 3580 WILSHIRE BLVD., 17TH FLOOR, LOS ANGELES, CA, 90010, US

(21) Appl. No.: 11/574,491

(22) PCT Filed: Aug. 30, 2005
(86) PCT No.: PCT/KR05/02873

§ 371 Date: Feb. 28, 2007

(30) Foreign Application Priority Data
Aug. 30, 2004 (KR) 10-2004-0068664

(51) Int. Cl. A61K 36/00; A61P 11/00

(52) U.S. Cl. 424/725

(57) ABSTRACT

The present invention relates to a herb composition for asthma maintenance therapy based on Chungsangboha-tang, which is known to be effective in asthma, capable of reducing the long-term suffering of asthma patients and improving their quality of lives and a manufacturing method thereof. The herbal composition of the present invention is characterized by comprising 5-18 parts by weight of Rehmanniae radix vaporata, 4-14 parts by weight oÊDioscoreae radix, 4-14 parts by weight of Corni fructus, 2-9 parts by weight of Pachyma hoelen, 2-9 parts by weight of Moutan radicis cortex, 2-9 parts by weight of Alismatis rhizoma, 2-7 parts by weight of Schizandrae fructus, 2-7 parts by weight of Aspargus cochinchinensis, 2-7 parts by weight of Liriopsis tuber, 2-7 parts by weight of Fritillariae bulbus, 2-7 parts by weight of Trichosantes kirilowii, 2-7 parts by weight of Prunus armeniacae linn, 2-7 parts by weight of Pinelliae rhizoma, 2-7 parts by weight oÊAurantii immaturus fructus, 2-7 parts by weight of Platycodi radix, 2-7 parts by weight of Scutellariae radix, 2-7 parts by weight of Coptidis rhizoma and 1-5 parts by weight of Glycyrrhizae radix.

### TECHNICAL FIELD

The present invention relates to a herb composition for asthma maintenance therapy and a manufacturing method thereof. More particularly, the present invention relates to a herb composition based on Chungsangboha-tang, a herb remedy known to be effective in treating asthma, and a manufacturing method thereof.

### BACKGROUND ART

Asthma is one of the commonest chronic diseases in the world, affecting about 10% of the world population. Its prevalence and severity are increasing each year. Not only in developed countries, asthma pauses a big problem in developing countries. In Korea, morbidity of asthma is 5 to 12.1% for children, 3.4% for adults and 12.7% for elderly people of 65 years or older. In spite of such high morbidity, recognition about asthma is insufficient and its severity is underestimated. Thus, we lack systematic prevention and therapy of the disease.

Bronchial asthma is a complex clinical syndrome characterized by reversible airway obstruction, bronchial hypersensitiveness, edema in airway and eosinophilic-lymphocytic inflammation. Clinically, bronchial asthma accompanies such symptoms as spasmodic dyspnea, coughing, wheezing, etc. These symptoms occur intermittently with periods of acute aggravation and silence interposed. With the development in immunology and molecular biology, the development of bronchoalveolar lavage and the pathophysiological researches on asthmatic bronchi, the opinion that “asthma is a reversible caused by airway hyperresponsitiveness” was corrected to one that “asthma is a chronic inflammatory disease in airway and the airway obstruction may be irreversible” since 1990.

In Oriental Medicine, bronchial asthma has been recognized as Hyocheon-Jeung, because of such symptoms as gasping and whooping.

Formerly, asthma was understood as hypersensitive reaction of bronchus, or abrupt constriction of bronchus, caused by specific allergy-inducing causes. However, recently, it was identified as chronic allergic inflammation of bronchus. That is, focus has been transferred to chronic bronchial inflammation rather than abrupt symptoms. Consequently, whereas focus was laid upon therapy of abrupt asthmatic spasm, it is now laid upon consistent therapy and control of chronic inflammation.

According to a special report by WHO in 2000, number of patients suffering from asthma around the world was 150 million and 180,000 people died of asthma every year. In 2001, 225,700 people died of asthma and experts estimate that about 300 million people around the world are suffering from light or severe asthma. During the past 20 years, incidence and prevalence of asthma increased in a lot of countries. However, the cause of the increase and the reason of difference of incidence and prevalence within and outside populations are not clear. Air pollution, automobile exhaust fumes, cigarette smoke, modern residence environment, hygiene, etc. are assumed to be the cause of increase of asthma. In the report, “Global Burden of Asthma,” the Global Initiative for Asthma (GINA) declared that the current therapy of asthma is a negative one not for prevention but for alleviation of acute symptoms and that prevalence of asthma is consistently increasing. In Korea, prevalence of asthma in children increased from 3 to 4% in early 1960s to 2 to 3 times. The prevalence in elderly people of 65 years or older is as high as 12.7%. As such, asthma is greatly affecting quality of life for both children and adults. According to a study performed by Shim Myung Ki, Choi In Sun et al. of Department of Allergy of Jeonnam National University Hospital on 994 asthmatic patients treated in emergency room from 1996 to 2002, number of patients treated in emergency room increased by 5.3 times during the 7-year period. According to “Asthma Insights and Reality in Asia Pacific (AIRIAP)” covering 3,200 asthmatic patients of eight Asian countries including Korea during the period of September to December 2000, Korea was second to Singapore with the ratio of asthmatic patients 6 to 7 years old being 13.3%.

Social, public and private cost related with asthma is enormous. Socioeconomic factor is essential in control of asthma in view of individual patients, medical experts and medical institutions. According to the 2000 special report by World Health Organization (WHO), medical and social cost of asthma is larger than the sum of the costs of tuberculosis and AIDS. In the U.S. alone, medical cost related with asthma amounts to about 6.4 billion dollars a year and decrease of parents' productivity due to children's asthma is estimated about 1 billion dollars. Uncontrolled asthma in a family member may hinder the other member's economic efficiency, while effective medical therapy gives economic advantage to the whole family members. It is reported that cost of medical therapy of asthma in a U.S. family amounts to 5.5 to 14.5% of total income. Primary care reduces cost compared with hospital therapy and emergency therapy costs more than planned therapy. While morbidity of asthma is increasing consistently because of aggravation of the diseases's severity, insufficient anti-inflammatory therapy, bronchus, over-dependence on bronchodilators, delayed hospital visit and so forth, it is reported that frequent and consistent outpatient therapy reduces hospitalization.

Guideline on the therapy of asthma was first proposed in 1995 (NHLBI/WHO Workshop Report: Global Strategy for Asthma Management and Prevention) by the GINA (Global Initiative for Asthma) program, which was begun in 1993, and was amended online in 2002 by the GINA Executive Committee (visit www.ginasthma.com). This guideline categorizes severity of asthma into four levels depending on symptoms and offers therapy for each level for long-term control and quick relief. The Korean Society of Allergology published a therapy guideline in 1998.

In 1992, international guideline on diagnosis and therapy of asthma was first formulated. In 1994, the Korean Society of Allergology presented a asthmatic therapy guideline for Koreans. In 1997, the NIH classification amended and complemented by the 2nd GINA expert group was presented. In 1998, the International Heart, Lung, and Blood Institute and the World Heath Organization published the GINA guideline for control of asthma.

Formerly, therapy of asthma focused on relieving asthmatic spasm, but now focus is brought into consistent therapy and control of chronic inflammation. Accordingly, the WHO, the Korean Society of Asthma and Allergology and so forth presented therapy guidelines that distinguish asthmatic spasm, aggravated condition and normal condition. They stress on “maintenance therapy” when there is no apparent symptom. According to the current GINA guideline, the purpose of asthma therapy is eradication or minimization of chronic asthmatic symptoms and securing of everyday life without any restriction caused by asthma. Even when a bronchus seems to have recovered from asthmatic spasm through drug therapy, etc. by short-term observation, long-term tracing of such patients shows it is not so. According to a study by Peter Lange et al., (they traced about 1,100 asthma patients and about 16,000 healthy people for 10 to 15 years), asthma patients showed about 1.5 times faster decline of lung function than healthy people. As for smokers, the rate of decline of lung function was about 2 times. That is, repetitive bronchial inflammation caused asthmatic spasm injures the bronchus and inner diameter of bronchus becomes narrower. Thus, as asthmatic symptom is alleviated and aggravated, lung function declines gradually. Consequently, maintenance therapy is important at the time when there is no apparent symptom.

Chungsangboha-tang is a prescription based on Chungsangboha pill, which was first mentioned in Dongeui Susebowon. Because it is effective in counterbalancing virile powers, smoothing lung, making phlegm and tempering fever, Chungsangboha-tang is used to treat lung diseases with the symptoms of flushing, asthma, coughing, grunting, etc. This medicine is utilized in treating chronic respiratory diseases. Chungsangboha-tang is prepared by adding Aspargus cochinchinensis and Liriopsis tuber, which are effective in invigorating the lung and discharging phlegm, Platycodi radix and Prunnus Armeniacae Linn, which are effective in clearing the lung, discharging phlegm and relieving cough, Schizandrae fructus, which is effective in improving concentration, invigorating energy, stopping cough and nourishing, Scutellariae radix and Coptidis rhizoma, which are effective in relieving heat from the lung and discharging phlegm, Fritillariae bulbus, which is effective in moisturizing and discharging phlegm, Aurantii Immaturus, which is effective in improving flow of vital energy and blood circulation, Pinelliae rhizoma, which is effective in relieving heat from the lung and improving flow of vital energy, and Glycyrrhizae radix and Trichosantes kirilowii, which are effective in counteracting and neutralizing poison, to Yukmijihwang-tang, a representative prescription for counterbalancing virile powers. The prescription of Chungsangboha pill appears at the last part of Susebowon written by Gong Jeong-Hyeon. He mentioned that Chungsangboha pill was effective in treating “an asthma patient with years-long, chronic symptoms of wheezing, frequent chillness, flushing, coughing, hard and obstructive phlegm.” The expression “chronic” seems that the asthma was quite severe. Yukmijihwang-tang is a decoction prescription in which Rehmanniae radix vaporata, Dioscoreae radix, Corni fructus, Pachyma hoelen, Moutan radicis cortex and Alismatis rhizoma with a proportion of 8:4:4:3:3:3. Originally, it was a prescription for children, but now, it is used for children and adults alike for counterbalancing virile powers. In general, it is prescribed for such symptoms as flushing or hot body temperature, rapid and strong pulse, thirstiness and desire for, especially, cold water, impatience with warmness, overly appetite, retarded growth of children and retarded intellectual development and is used to treat scrofulousness of children, chronic liver diseases, malnutrition, congenital weakness, etc. It is also prescribed for diabetes. Yukmijihwang-tang is reported to reduce outbreak of stomach cancer, reduce severe proliferation of esophagus epithelial cells, which is the early stage of esophagus cancer, and inhibit lung and lymphocyte tumors. Also, there is a report that when the medicine was administered to white rats with sexual dysfunction for 7 to 14 days, sexual function was improved outstandingly. In addition, it was confirmed to have a significant clinical improving effect for diabetes. However, remedial or preventive effect of Yukmijihwang-tang on asthma is not known yet.

Since bronchial asthma is a chronic disease which recurs frequently, long-term taking of herbal medicine is inevitable. However, there is no report about the consistency of the effect of herbal medicine when administration thereof was interrupted once the disease was alleviated.

Recently, the WHO, the Korean Society of Allergology and so forth presented therapy guidelines that distinguish asthmatic spasm, aggravated condition and normal condition. They stress on “maintenance therapy” when there is no apparent symptom. As for bronchial asthma, effect of herbal medicine is reported based on several clinical trials. Since bronchial asthma is a chronic disease which recurs frequently, repeated, long-term taking of herbal medicine is required, but it is not an easy matter considering the cost.

Until now, a prescription for aiding asthma therapy or preventing asthmatic spasm, which can be used for an asthma patient who was treated with herbal medicine and was improved therefrom, was not developed.

The present inventors developed a herbal composition for maintenance therapy of asthma and confirmed that it shows remedial effect for the four patient groups according to the GINA guideline.

### DISCLOSURE OF INVENTION

### Technical Problem

It is an object of the present invention to provide a herbal composition for maintenance therapy of asthma based on the prescription of Chungsangboha-tang, which is known to be effective in treating asthma, capable of reducing the long-term suffering of asthma patients and improving their quality of lives and a manufacturing method thereof.

### Technical Solution

The herbal composition for maintenance therapy of asthma in accordance with the present invention comprises 5˜parts by weight of Rehmanniae radix vaporata, 4˜14 parts by weight of Dioscoreae radix, 4˜parts by weight of Corni fructus, 2˜9 parts by weight of Pachyma hoelen, 2˜9 parts by weight of Moutan radicis cortex, 2˜9 parts by weight of Alismatis rhizoma, 2˜7 parts by weight of Schizandrae fructus, 2˜7 parts by weight of Aspargus cochinchinensis, 2˜7 parts by weight of Liriopsis tuber, 2˜7 parts by weight of Fritillariae bulbus, 2˜7 parts by weight of Trichosantes kirilowii, 2˜7 parts by weight of Prunus armeniacae linn, 2˜7 parts by weight of Pinelliae rhizoma, 2˜7 parts by weight of Aurantii immaturus fructus, 2˜7 parts by weight of Platycodi radix, 2˜7 parts by weight of Scutellariae radix, 2˜7 parts by weight of Coptidis rhizoma and 1˜5 parts by weight of Glycyrrhizae radix.

The method of preparing the herbal composition of the present invention comprises: (1) a preparation process of mixing 5˜18 parts by weight of Rehmanniae radix vaporata, 4˜14 parts by weight of Dioscoreae radix, 4˜14 parts by weight of Corni fructus, 2˜9 parts by weight of Pachyma hoelen, 2˜parts by weight of Moutan radicis cortex, 2˜9 parts by weight of Alismatis rhizoma, 2˜7 parts by weight of Schizandrae fructus, 2˜7 parts by weight of Aspargus cochinchinensis, 2˜7 parts by weight of Liriopsis tuber, 2˜7 parts by weight of Fritillariae bulbus, 2˜7 parts by weight of Trichosantes kirilowii, 2˜7 parts by weight of Prunus armeniacae linn, 2˜7 parts by weight of Pinelliae rhizoma, 2˜7 parts by weight of Aurantii immaturus fructus, 2˜7 parts by weight of Platycodi radix, 2˜7 parts by weight of Scutellariae radix, 2˜7 parts by weight of Coptidis rhizoma and 1˜5 parts by weight of Glycyrrhizae radix crushing the mixture; (2) an extraction process of extracting the crushed mixture with hot water; (3) a filtration process of filtering the extract and separating the remainder aliquot; (4) a concentration process of concentrating the remainder aliquot; and (5) a drying process of drying the concentrate.

In the extraction process, extraction is performed for 1˜3 hours using hot water of 80-100° C.

### Advantageous Effects

The present invention is advantageous in offering a herb composition for asthma maintenance therapy based on the prescription of Chungsangboha-tang, which is known to be effective in treating asthma, capable of reducing the long-term suffering of asthma patients and improving their quality of lives and a manufacturing method thereof.

### BEST MODE FOR CARRYING OUT THE INVENTION

Hereunder is given a detailed description of the present invention.

The herbal composition for maintenance therapy of asthma in accordance with the present invention is characterized by comprising 5˜18 parts by weight of Rehmanniae radix vaporata, 4˜14 parts by weight of Dioscoreae radix, 4˜14 parts by weight of Corni fructus, 2˜9 parts by weight of Pachyma hoelen, 2˜9 parts by weight of Moutan radicis cortex, 2˜9 parts by weight of Alismatis rhizoma, 2˜7 parts by weight of Schizandrae fructus, 2˜7 parts by weight of Aspargus cochinchinensis, 2˜7 parts by weight of Liriopsis tuber, 2˜7 parts by weight of Fritillariae bulbus, 2˜7 parts by weight of Trichosantes kirilowii, 2˜7 parts by weight of Prunus armeniacae linn, 2˜7 parts by weight of Pinelliae rhizoma, 2˜7 parts by weight of Aurantii immaturus fructus, 2˜7 parts by weight of Platycodi radix, 2˜parts by weight of Scutellariae radix, 2˜7 parts by weight of Coptidis rhizoma and 1˜parts by weight of Glycyrrhizae radix.

Each constituent of the herbal composition is known to have the following medicinal effects.

Rehmanniae radix vaporata improves memory and learning ability, reduces blood-sugar level, stimulates heart and promotes urination. Also, it has liver function protecting, antibacterial, nourishing and antihistamine effects.

Dioscoreae radix is effective in discharging phlegm and fighting inflammation.

Corni fructus has transient blood pressure drop, anticancer and antibacterial effects, promotes urination and helps digestion of proteins.

Pachyma hoelen is effective in hemolysis prevention, therapy of digestive organ diseases, memory enhancement, prevention of mutation, prevention of cancer through antioxidation activity, promotion of urination and tranquilization.

Moutan radicis cortex is effective in thrombosis prevention, prevention of mutation through antioxidation activity, tranquilization and hypnosis, pain alleviation, blood pressure drop, prevention of leg edema and fighting bacteria.

Alismatis rhizoma has anticancer activity, anti-inflammatory activity through prevention of nitrogen monoxide production and anti-allergic effect.

Schizandrae fructus has liver protecting and regenerating activity, anti-cancer activity, brain cell protecting activity and memory loss recovery effect, etc.

Aspargus cochinchinensis is effective in preventing liver cancer, counteracting poison in the liver and discharging phlegm.

Liriopsis tuber is effective in relieving cough and discharging phlegm through de-composition of proteins in the phlegm, protecting bronchus through anti-inflammatory activity, treating coronary artery diseases, etc.

Fritillariae bulbus has anti-cancer activity, blood pressure drop activity, etc. and is used to treat coughing, phlegm, pneumonia, tonsillitis, throat pain, bronchitis and thirst. It is also applied on cuts, festers and stings.

Trichosantes kirilowii has anti-cancer activity, antiphlogistic activity, phlegm discharging activity, anti-bacterial activity, etc.

Prunus armeniacae linn has effect on the liver and the large intestine. It is effective in treating coughing, phlegm, asthma, etc. Rich in fat, Prunnus Armeniacae Linn is good for constipation and helps digestion by promoting peristalsis of the stomach and intestines. In addition, it is known to have anti-mutation activity.

Pinelliae rhizoma relieves coughing and vomiting and has stabilizing and spasm-inhibiting activities.

Aurantii immaturus fructus improves flow of vital energy, alleviates congestion and improves digestive function. It is also known to help contraction of the womb, improve motion of the stomach and intestines, stimulate heart and promotes urination.

Platycodi radix has fat removal, liver protection, cough relieving and phlegm discharging activities.

Scutellariae radix is used to treat heart attack, promote urination, relieve antiphlogistic fever, treat congestive inflammation and treat sclerosis. It is also used for pyemia, stomachache, sore, fever, inflammation, hemorrhoids, miscarriage, bleeding, jaundice, erysipelas, vomiting, diarrhea, etc. It is also used as bathing perfume.

Coptidis rhizoma has antibacterial activity, fever relieving activity, anti-inflammatory activity, anti-cancer activity, pimple treating activity, cholesterol reducing activity, nerve fiber growth promoting activity, etc.

Glycyrrhizae radix has heart protecting activity, anti-cancer activity, antithrombolytic activity, poison counterbalancing activity, antispasmodic activity, gastric acid secretion inhibiting activity and phlegm discharging activity.

If the content of Rehmanniae radix vaporata is below 5 parts by weight, anti-histamine effect decreases, so that effective therapy of asthma may not be attained. Otherwise, if it exceeds 18 parts by weight, such side effects as indigestion may occur.

Preferably, Dioscoreae radix, which has phlegm discharging activity and anti-inflammatory activity, is comprised in more than 4 parts by weight. However, if its content exceeds 14 parts by weight, such side effects as indigestion may occur.

Corni fructus offers effective anti-inflammatory effect without such side effects as indigestion when it is comprised in 4˜14 parts by weight.

Pachyma hoelen inhibits asthmatic inflammation through effective antioxidation effect, when comprised in 2˜9parts by weight.

If the content of Moutan radicis cortex is below 2 parts by weight, allergic asthma may not be treated effectively. Otherwise, if it exceeds 9 parts by weight, such side effects as interfering the central nerve system may occur.

Preferably, Alismatis rhizoma is comprised in 2-9 parts by weight in order to effectively prevent aggravation of asthma caused by bronchial inflammation due to excessive nitrogen monoxide formation while preventing such side effects as indigestion and gastric bleeding.

If the content of Schizandrae fructus is below 2 parts by weight, coughing caused by asthma cannot be effectively relieved. Otherwise, if it exceeds 7 parts by weight, allergic reaction may occur or asthma may be aggravated.

Aspargus cochinchinensis effectively discharges phlegm while preventing such side effects as chronic inflammation, when comprised in 2˜7 parts by weight.

Liriopsis tuber is effective in relieving cough by decomposing proteins in the phlegm, discharging phlegm and protecting bronchus through anti-inflammatory activity, when comprised in 2˜7 parts by weight.

If the content of Fritillariae bulbus is below 2 parts by weight, asthma may not be treated effectively because of insufficient anti-inflammatory activity. Otherwise, if it exceeds 7 parts by weight, such side effects as indigestion may occur.

Trichosantes kirilowii is effective in treating asthma through phlegm discharging activity and anti-inflammatory activity, when comprised in more than 2 parts by weight. However, if its content exceeds 7 parts by weight, immune prevention, allergic reactions, etc. may occur because of its toxicity.

Prunus armeniacae linn effectively discharges phlegm and fights inflammation while preventing such side effects as cyanide poisoning, when comprised in 2-7 parts by weight.

Pinelliae rhizoma effectively relieves coughing and phlegm while preventing such side effects as miscarriage and uterine bleeding, when comprised in 2-7 parts by weight.

If the content of Aurantii immaturus fructus is below 2 parts by weight, allergic asthma diseases may not be treated effectively. Otherwise, if it exceeds 7 parts by weight, such side effects as indigestion may occur.

Platycodi radix effectively prevents phlegm and inflammation in respiratory organs caused by asthma while preventing such side effects as indigestion and gastric bleeding, when comprised in 2˜7 parts by weight.

If the content of Scutellariae radix is below 2 parts by weight, asthma-induced inflammation may not be relieved effectively. Otherwise, if it exceeds 7 parts by weight, gastrointestinal disturbance may occur.

Coptidis rhizoma effectively prevents inflammation in respiratory organs while preventing such side effects as indigestion, when comprised in 2-7 parts by weight.

And, Glycyrrhizae radix effectively relieves cough by decomposing proteins in the phlegm, discharges phlegm and protects bronchus through anti-inflammatory activity, when comprised in 1˜5 parts by weight.

The method for preparing the herbal composition for treating asthma in accordance with the present invention is characterized by comprising: (1) a preparation process of mixing 5˜18 parts by weight of Rehmanniae radix vaporata, 4˜14 parts by weight of Dioscoreae radix, 4˜14 parts by weight of Corni fructus, 2˜9 parts by weight of Pachyma hoelen, 2˜9 parts by weight of Moutan radicis cortex, 2˜9 parts by weight of Alismatis rhizoma, 2˜7 parts by weight of Schizandrae fructus, 2˜7 parts by weight of Aspargus cochinchinensis, 2˜7 parts by weight of Liriopsis tuber, 2˜7 parts by weight of Fritillariae bulbus, 2˜7 parts by weight of Trichosantes kirilowii, 2˜7 parts by weight of Prunus armeniacae linn, 2˜7 parts by weight of Pinelliae rhizoma, 2˜7 parts by weight of Aurantii immaturus fructus, 2˜7 parts by weight of Platycodi radix, 2˜7 parts by weight of Scutellariae radix, 2˜7 parts by weight of Coptidis rhizoma and 1-5 parts by weight of Glycyrrhizae radix and crushing the mixture; (2) an extraction process of extracting the crushed mixture with hot water; (3) a filtration process of filtering the extract and separating the remainder aliquot; (4) a concentration process of concentrating the remainder aliquot; and (5) a drying process of drying the concentrate. Here, each component of the herbal composition offers the same effect as described above.

In the preparation process (1), the components of the herbal composition of the present invention are mixed and crushed so that the active ingredients are extracted well. In the extraction process (2), the mixture is extracted with water. The extraction is performed for 1˜3 hours using hot water of 80˜100° C. The extraction using hot water is a process well known to one skilled in the art and can be widely used to extract active ingredients from the herbal composition for convenience in taking. If the temperature of the hot water is below 80° C., extraction may be insufficient. Otherwise, if it exceeds 100° C., active ingredients may be evaporated or the volume of the resultant decoction may be inadequate. However, it is easily understood by one skilled in the art that the temperature may vary depending upon the amount of source materials, heat source of extraction and container, etc. In the filtration process (3), active ingredients are separated from the portion remaining after the extraction as remainder aliquot. The resultant remainder aliquot is concentrated in the succeeding concentration process and dried in the following drying process by spray drying, lyophilization, etc. As a result, moisture is removed and only active ingredients are granulated for convenience of taking. The herbal composition may be extracted by common method using at least one solvent selected from a group consisting of water, C₁-C₅ alcohol and an aqueous alcohol solution.

### MODE FOR THE INVENTION

Hereinafter, the present invention is described referring to the preferred embodiments. However, the following examples are only for the understanding of the invention and they shall not be construed as limiting the scope of the invention.

### EXAMPLE

8 g of Rehmanniae radix vaporata, 6 g of Dioscoreae radix, 6 g of Corni fructus, 4 g of Pachyma hoelen, 4 g of Moutan radicis cortex, 4 g of Alismatis rhizoma, 3 g of Schizandrae fructus, 3 g of Aspargus cochinchinensis, 3 g of Liriopsis tuber, 3 g of Fritillariae bulbus, 3 g of Trichosantes kirilowii, 3 g of Prunus Armeniacae Linn, 3 g of Pinelliae rhizoma, 3 g of Aurantii Immaturus, 3 g of Platycodi radix, 3 g of Scutellariae radix, 3 g of Coptidis rhizoma and 2 g of Glycyrrhizae radix were mixed and prepared into an extract. Each constituent of the herbal medicine composition was crushed to obtain powder. Powder of each constituent was mixed, as described above, and put in a flask filled with distilled water. Extraction was performed for 1 hour using hot water of 100° C. Then, remainder aliquot filtered with gauze was concentrated with a vacuum filter and dried with a spray drier to obtain powder.

### Testing Example 1

The following test was performed with the prepared herbal composition extract to confirm its maintenance therapy effect on asthma of herbal composition according to the present invention.

1. Subjects and Test Method

1) Subjects

Of the patients who volunteered the clinical test performed at Kyung Hee University Hospital, 27 patients who had been diagnosed as bronchial asthma with typical clinical symptoms (intermittent reversible dyspnea, coughing, phlegm, wheezing, suppressed chest, etc.) and with the forced expiratory volume at one second (FEV_{1.0}) at the PFT test increasing 12% or more after use of β-2 bronchodilator were selected. The herbal composition extract of Example was administered to the patients for 4 weeks. Tracing was performed 4 weeks after stopping the administration. All the subjects had been fully informed of the purpose of the clinical research and signed written consents.

2) Herbs

The herbal composition extract of Example was administered to the patient group for 4 weeks, three times a day 1 hour after each meal, one pack at a time. The herbal composition extract was prepared from the herbs purchased from Kyung Hee University Hospital into 6 g per unit.

3) Testing

Questionnaire survey and lung performance test were performed for 3 times—prior to administration of the herbal composition extract, after 4 weeks of administration and 4 weeks after stopping the administration. “Quality of Life Questionnaire for Korean Asthmatics (QLQAKA)” approved by the Korean Society of Allergology was used for the survey. Lung performance test was conducted at Kyung Hee Medical Center. FEV_{1.0}, forced vital capacity (FVC) and peak expiratory flow rate (PEFR) were measured for 3 times using a spirometer (Sensorimedics, U.S.A).

4) Statistical Therapy

Change in the patient group before administration of the herbal composition extract, after 4 weeks of administration and 4 weeks after stopping the administration was examined by the Wilcoxon signed-ranks test. Each result was presented as mean +standard deviation. Statistical test was performed with the SPSS 11.0 software. Statistical significance of each run was p<0.05.

5) Result Analysis

5-1) Characteristics of Patient Group

The patient group consisted of 14 men and 13 women, 48.561±10.54 years old on the average.

5-2) Change of Lung Performance

The test result is as follows. As seen in Table 1 below, FVC increased from 84.33±15.43% (before administration) to 85.52±14.06% (after 4 weeks of administration) to 87.78±12.73% (4 weeks after stopping the administration). FEV_{1.0} increased from 62.38±21.80% (before administration) to 73.89±18.59% (after 4 weeks of administration) to 74.04±17.65% (4 weeks after stopping the administration). PEFR increased from 64.85±18.89% (before administration) to 73.11±21.38% (after 4 weeks of administration) to 75.52±21.61% (4 weeks after stopping the administration).

TABLE 1

| | | 4 weeks after | |
|---|---|---|---|
| | Before admin- | stopping admin- | |
| PFT | istration | istration | p-value²⁾ |
| FVC (%) | 84.33 ± 15.43 | 87.78 ± 12.73¹⁾ | ns |
| FEV_{1.0} (%) | 62.38 ± 21.80 | 74.04 ± 17.65 | ns |
| PEFR (%) | 64.85 ± 18.89 | 75.52 ± 21.61 | ns |
| | | 4 weeks after | |
| | After 4 weeks of | stopping admin- | |
| PFT | administration | istration | p-value²⁾ |
| FVC (%) | 85.52 ± 14.06 | 87.78 ± 12.73¹⁾ | ns |
| FEV_{1.0} (%) | 73.89 ± 18.59 | 74.04 ± 17.65 | ns |
| PEFR (%) | 73.11 ± 21.38 | 75.52 ± 21.61 | ns |

¹⁾FVC, FEV_{1.0}, PEFR = mean ± standard deviation.
²⁾Statistical therapy by Wilcoxon signed-ranks test.
ns: Non-significant.

5-3) QLQAKA Survey Result

The QLQAKA survey result is as follows. As seen in Table 2 below, the QLQAKA value increased from 3.28±0.68 (before administration) to 3.77±0.84 (after 4 weeks of administration) to 3.68±0.79 (4 weeks after stopping the administration) (p<0.05).

TABLE 2

| | | 4 weeks after | |
|---|---|---|---|
| | Before admin- | stopping admin- | |
| QLQAKA | istration | istration | p-value²⁾ |
| Patient group | 3.28 ± 0.68 | 3.68 ± 0.79¹⁾ | ns |
| | | 4 weeks after | |
| | After 4 weeks of | stopping admin- | |
| QLQAKA | administration | istration | p-value²⁾ |
| Patient group | 3.77 ± 0.84 | 3.68 ± 0.79¹⁾ | ns |

¹⁾QLQAKA = mean ± standard deviation.
²⁾Statistical therapy by Wilcoxon signed-ranks test.
ns: Non-significant.

As seen above, 4 weeks of administration of the herbal composition extract increased PFT and QLQAKA but the increase was statistically non-significant.

There was no significant change in QLQAKA and PFT after 4 weeks of administration and 4 weeks after stopping the administration.

With 4 weeks of administration of the herbal composition extract, FEV_{1.0} and PEFR increased. QLQAKA also increased.

FEV_{1.0} and PEFR, which showed significant increase during the 4 weeks of administration of the herbal composition extract, did not show significant change after stopping the administration. The same was true of QLQAKA. This shows that the administration of the herbal composition extract offered consistent effect on the quality of life of asthma patients. It is considered that re-administration is required after 4 weeks to 3 months stopping the administration.

### Testing Example 2

The following test was performed with the herbal composition extract prepared in Example.

1. Subjects and Test Method

1) Subjects

Test was performed on the 27 patients who had been tested in Testing Example 1. The herbal composition extract was administered for 4 weeks after Testing Example 1 had finished. All the subjects had been fully informed of the purpose of the clinical research and signed written consents.

2) Herbs

The herbal composition extract of Example was administered to the patient group for 4 weeks, three times a day 1 hour after each meal, one pack at a time. The herbal composition extract was prepared from the herbs purchased from Kyung Hee University Hospital into 6 g per unit.

3) Testing

Questionnaire survey was performed for 3 times—prior to administration of the herbal composition extract, after 2 weeks of administration and after 4 weeks of administration. Lung performance test were performed for 2 times—prior to administration of the herbal composition extract and after 4 weeks of administration.

“Quality of Life Questionnaire for Korean Asthmatics (QLQAKA)” approved by the Korean Society of Allergology was used for the survey (Park Jung-Won, Cho Yoo-Sook, Nam Dong-Ho, Kim Yoon-Geun, Kim Dong-Ki et al., Inter-Disciplinary Research for Evaluation of Usefulness of Quality of Life Questionnaire for Korean Adult Bronchial Asthmatics, Asthma and Allergic Diseases 2000; 20(3): 467-479). Lung performance test was conducted at Kyung Hee Medical Center. FEV_{1.0}, forced vital capacity (FVC) and peak expiratory flow rate (PEFR) were measured for 2 times using a spirometer (Sensorimedics, U.S.A).

4) Statistical Therapy

Change in the patient group before and after administration of the herbal composition extract was examined by the paired t-test. The patient group was subdivided depending on the severity of asthma and change before and after administration of the herbal composition extract was examined by the Wilcoxon signed-ranks test. Each result was presented as mean±standard deviation. Statistical test was performed with the SPSS 11.0 software. Statistical significance of each run was p<0.05.

5) Result Analysis

5-1) Characteristics of Patient Group

The patient group consisted of 14 men and 13 women, 48.56±10.54 years old on the average. The patient group was subdivided according to the GINA category (Step 2: 4, Step 3: 12, Step 4: 11). Step 2 patients were 45.50±13.03 years old and showed FVC of 100.75±8.14%, FEV_{1.0} of 93.00±9.42% and PEFR of 99.75±13.89%. Step 3 patients were 47.42±11.91 years old and showed FVC of 85.42±11.49%, FEV_{1.0} of 69.92±11.48% and PEFR of 72.75±22.60%. Step 4 patients were 50.91±8.38 years old and showed FVC of 85.64±13.32%, FEV_{1.0} of 71.64±21.63% and PEFR of 69.73±17.71%. On the whole, the patients were 48.56±10.54 years old and showed FVC of 87.78±12.73%, FEV_{1.0} of 74.04±17.65% and PEFR of 75.52±21.61%.

5-2) Change of Lung Performance

The test result is as follows. As seen in Table 3 and Table 4 below, FVC of the 27 patients increased from 87.78±12.73% to 88.26±12.37%. FEV_{1.0} increased from 74.04±17.65% to 75.48±17.44%. PEFR increased significantly from 75.52±21.61% to 80.22±23.62%.

For Step 2 patients, FVC decreased 100.75±8.14% to 99.00±5.60% and FEV_{1.0} decreased from 93.00±9.42% to 91.75±7.89%, but the decrease was statistically non-significant. PEFR increased from 99.75±13.89% to 103.00±17.05%, but the increase was statistically non-significant.

For Step 3 patients, FVC increased from 85.42±11.49% to 86.58±13.28% and FEV_{1.0} increased from 69.92±11.48% to 72.17±12.43%, but the increase was statistically non-significant. PEFR increased significantly from 72.75±22.60% to 78.67±25.16% (p<0.05).

For Step 4 patients, FVC increased from 85.64±13.32% to 86.18±11.84%, FEV_{1.0} increased from 71.64±21.63% to 73.18±21.84% and PEFR increased from 69.73±17.71% to 73.64±20.23%, but the increase was statistically non-significant.

TABLE 3

| | Before admin- | | |
|---|---|---|---|
| PFT (n = 27) | istration | After 4 weeks | p-value²⁾ |
| FVC (%) | 87.78 ± 12.73¹⁾ | 88.26 ± 12.37 | ns |
| FEV_{1.0} (%) | 74.04 ± 17.65 | 75.48 ± 17.44 | ns |
| PEFR (%) | 75.52 ± 21.61 | 80.22 ± 23.62 | 0.002 |

¹⁾FVC, FEV_{1.0}, PEFR = mean ± standard deviation.
²⁾Statistical therapy by paired t -test.
ns: Non-significant.

TABLE 4

| | | Before admin- | After admin- | |
|---|---|---|---|---|
| Group | | istration | istration | p-value²⁾ |
| Step 2 | FVC (%) | 100.75 ± 8.14¹⁾ | 99.00 ± 5.60 | ns |
| (n = 4) | FEV_{1.0} (%) | 93.00 ± 9.42 | 91.75 ± 7.89 | ns |
| | PEFR (%) | 99.75 ± 13.89 | 103.00 ± 17.05 | ns |
| Step 3 | FVC (%) | 85.42 ± 11.49 | 86.58 ± 13.28 | ns |
| (n = 12) | FEV_{1.0} (%) | 69.92 ± 11.48 | 72.17 ± 12.43 | ns |
| | PEFR (%) | 72.75 ± 22.60 | 78.67 ± 25.16 | 0.016 |
| Step 4 | FVC (%) | 85.64 ± 13.32 | 86.18 ± 11.84 | ns |
| (n = 11) | FEV_{1.0} (%) | 71.64 ± 21.63 | 73.18 ± 21.84 | ns |
| | PEFR (%) | 69.73 ± 17.71 | 73.64 ± 20.23 | ns |

¹⁾FVC, FEV_{1.0}, PEFR = mean ± standard deviation.
²⁾Statistical therapy by Wilcoxon signed-ranks test.
ns: Non-significant.

5-3) QLQAKA Survey Result

As seen in Table 5 and Table 6 below, the QLQAKA value for the 27 patients increased from 3.68±0.79 (before administration) to 3.80±0.83 (after 2 weeks; significant increase; p<0.05) to 3.84±0.87 (statistically non-significant increase).

TABLE 5

| | Before | | | | |
|---|---|---|---|---|---|
| QLQAKA | administration | After 2 weeks | p-value²⁾ | After 4 weeks | p-value²⁾ |
| Patient group (n = 27) | 3.68 ± 0.79¹⁾ | 3.80 ± 0.83 | 0.017 | 3.84 ± 0.87 | ns |

¹⁾QLQAKA = mean ± standard deviation.
²⁾Statistical therapy by paired t-test.
ns: Non-significant.

TABLE 6

| | Before | | | | |
|---|---|---|---|---|---|
| QLQAKA | administration | After 2 weeks | p-value²⁾ | After 4 weeks | p-value²⁾ |
| Step 2 (n = 4) | 4.13 ± 0.29¹⁾ | 4.43 ± 0.41 | ns | 4.09 ± 0.78 | ns |
| Step 3 (n = 12) | 3.86 ± 0.67 | 3.90 ± 0.78 | ns | 4.12 ± 0.80 | 0.021 |
| Step 4 (n = 11) | 3.32 ± 0.90 | 3.47 ± 0.88 | 0.041 | 3.44 ± 0.90 | ns |

¹⁾QLQAKA = mean ± standard deviation.
²⁾Statistical therapy by Wilcoxon signed-ranks test.
ns: Non-significant.

For Step 2 patients, the QLQAKA value increased from 4.13±0.29 (before administration) to 4.43±0.41 (after 2 weeks) and decreased to 4.09±0.78, but the change was statistically non-significant. For Step 3 patients, the QLQAKA value increased from 3.86±0.67 to 3.90±0.78 after 2 weeks, which was statistically non-significant, and increased significantly to 4.12±0.80 after 4 weeks (p<0.05). For step 4 patients, the QLQAKA value increased significantly from 3.32±0.90 to 3.47±0.88 after 2 weeks (p<0.05) and increased to 3.44±0.90 after 4 weeks, but the increase was statistically non-significant.

As seen in Table 3 and Table 4, all of PEFR, FVC and FEV_{1.0} increased, but the increase was statistically non-significant. 4 weeks of administration of the herbal composition extract seems to have improved lung performance of the asthmatics. The quality of life increased both after 2 weeks and after 4 weeks, but only the increase after 2 weeks was statistically significant. To sum up, quality of life, which decreased after stopping administration of the herbal composition extract, although statistically non-significantly, was confirmed to improve quickly with its administration.

The GINA guideline categorizes asthma patients into 4 groups—Step 1 for intermittent minor asthma, Step 2 for continued minor asthma, Step 3 for continued moderate asthma and Step 4 for continued severe asthma. More severe asthmatics were older and showed lower quality of life, FEV_{1.0} and PEFR. Significant improvement of lung performance was observed in Step 3 patients.

As for quality of life, Step 3 patients showed significant improvement after 4 weeks and Step 4 patients showed significant improvement after 2 weeks. Step 2 patients showed increase of the QLQAKA value after 2 weeks and decrease after 4 weeks, but the change was statistically non-significant. This shows that the herbal composition extract improved or at least sustained quality of life of patients with continued asthma symptoms of Step 2 or more severe.

As described above, the herbal composition extract of the present invention improved or at least sustained lung performance and quality of life of patients with continued asthma symptoms of Step 2 or more severe, when re-administered after stopping 4 weeks of administration of the extract. Thus, it may be able to solve the problem of inconvenience of taking and storing medicinal decoction. Considering that asthma is a chronic disease with frequent recurrence and spasm, the present invention is advantageous in that it can prevent aggravation of the asthmatic symptom by maintaining and improving lung performance and quality of life. Accordingly, the herbal composition extract of the present invention is effective for long-term maintenance of improved lung performance and quality of life. When administration of the extract is stopped, it seems that it should be re-administered within 4 weeks to 3 months to attain continued improvement of quality of life.

To conclude, when the herbal composition extract of the present invention was re-administered after 4 weeks stopping administration, the 27 bronchial asthmatics showed improved or at least sustained QLQAKA and PFT. In detail, a continued improvement of quality of life was observed for continued asthmatics of Step 3 or more severe. Also, significant improvement of lung performance was observed for continued asthmatics of Step 3 or more severe. Thus, the herbal composition extract of the present invention seems to be clinically useful for long-term management of asthmatics and it seems that it should be re-administered within 4 weeks to 3 months after stopping its administration.

As apparent from the above description, the herbal composition for maintenance therapy of asthma in accordance with the present invention is very helpful to asthmatics as it can improve their quality of life.

While the present invention has been described in detail with reference to the preferred embodiments, those skilled in the art will appreciate that various modifications and substitutions can be made thereto without departing from the spirit and scope of the invention as set forth in the appended claims.

1. A herbal composition for maintenance therapy of asthma comprising 5˜18 parts by weight of Rehmanniae radix vaporata, 4˜14 parts by weight of Dioscoreae radix, 4˜14 parts by weight of Corni fructus, 2˜9 parts by weight of Pachyma hoelen, 2˜9 parts by weight of Moutan radicis cortex, 2˜9 parts by weight of Alismatis rhizoma, 2˜7 parts by weight of Schizandrae fructus, 2˜7 parts by weight of Aspargus cochinchinensis, 2˜7 parts by weight of Liriopsis tuber, 2˜7 parts by weight of Fritillariae bulbus, 2˜7 parts by weight of Trichosantes kirilowii, 2˜7 parts by weight of Prunus armeniacae linn, 2˜7 parts by weight of Pinelliae rhizoma, 2˜7 parts by weight of Aurantii immaturus fructus, 2˜7 parts by weight of Platycodi radix, 2˜7 parts by weight of Scutellariae radix, 2˜7 parts by weight of Coptidis rhizoma and 1˜5 parts by weight of Glycyrrhizae radix.

2. A solvent extract obtained by extracting the herbal composition of claim 1 with at least one solvent selected from a group consisting of water, C₁-C₅ alcohol and an aqueous alcohol solution.

3. An extract in the powder from obtained by drying the solvent extract of claim 2.

4. A manufacturing method of a herbal composition for maintenance therapy of asthma, comprising:
(1) a preparation process of mixing 5˜18 parts by weight of Rehmanniae radix vaporata, 4˜14 parts by weight of Dioscoreae radix, 4˜14 parts by weight of Corni fructus, 2˜9 parts by weight of Pachyma hoelen, 2˜9 parts by weight of Moutan radicis cortex, 2˜9 parts by weight of Alismatis rhizoma, 2˜7 parts by weight of Schizandrae fructus, 2˜7 parts by weight of Aspargus cochinchinensis, 2˜7 parts by weight of Liriopsis tuber, 2˜7 parts by weight of Fritillariae bulbus, 2˜7 parts by weight of Trichosantes kirilowii, 2˜7 parts by weight of Prunus armeniacae linn, 2˜7 parts by weight of Pinelliae rhizoma, 2˜7 parts by weight of Aurantii immaturus fructus, 2˜7 parts by weight of Platycodi radix, 2˜7 parts by weight of Scutellariae radix, 2˜7 parts by weight of Coptidis rhizoma and 1˜5 parts by weight of Glycyrrhizae radix and crushing the mixture;
(2) an extraction process of extracting the crushed mixture with hot water;
(3) a filtration process of filtering the extract and separating the remainder aliquot;
(4) a concentration process of concentrating the remainder aliquot; and
(5) a drying process of drying the concentrate.
